(19) United States
(12) Patent Application Publication
(10) Pub. No.: US 2014/0350594 A1
(43) Pub. Date: Nov. 27, 2014

(54) METHOD AND DEVICE FOR REDUCING AND PREVENTING SNORING AND SLEEP APNEA

(71) Applicant: Melissa Smith-McGinity, Smithfield, RI (US)

(72) Inventor: Melissa Smith-McGinity, Smithfield, RI (US)

(21) Appl. No.: 14/282,133

(22) Filed: May 20, 2014

Related U.S. Application Data
(60) Provisional application No. 61/825,557, filed on May 21, 2013.

(51) Int. Cl. A61F 5/56

(52) U.S. Cl. CPC A61F 5/56
USPC 606/201

(57) ABSTRACT

A method and device of reducing and preventing snoring and sleep apnea is disclosed. The device includes a belt of sufficient length to wrap around a person's torso and of sufficient width to overlap the person's abdomen and a fastening and tightening mechanism, such as a strap and buckle, attached to the belt configured and arranged to tighten the belt. The belt is positioned around a person's abdomen. The belt is then tightened with the fastening and tightening mechanism, sufficiently to apply pressure to the person's abdomen, applying specific pressure beneath a person's diaphragm. The fastening and tightening mechanism is then fastened to prevent loosening of the belt.

### CROSS-REFERENCE TO RELATED APPLICATION

This patent document claims priority to earlier filed U.S. Provisional Patent Application No. 61/825557, filed May 21, 2013, the entire contents of which are incorporated herein by reference.

### BACKGROUND

1. Technical Field

The present patent document relates generally to sleep disorders and treatments and more particular to a method and device for reducing and preventing snoring and sleep apnea.

2. Background of the Related Art

Snoring and sleep apnea are problems that are highly disruptive not only to the person having the problem, but to all those living with them. The problems of snoring and sleep apnea are extremely well-documented, causing lost sleep and other adverse health effects in those effected.

Prior art methods often focus on the airway and/or sleeping position of the patient. However, devices that focus on a person's airway include cumbersome or uncomfortable mouth guards or breathing masks that can also be expensive as well. Devices that focus on a person's sleeping position can also prove cumbersome and uncomfortable as well as they prevent a person from sleeping on their back or use specialty pillows that are uncomfortable as well

Accordingly, there is always a need for new methods and devices for treating snoring and sleep apnea that allows a person to sleep in a position they prefer without uncomfortable gear.

### SUMMARY

The present invention solves the problems of the prior art by providing a belt with a fastening and tightening mechanism configured and arranged to apply pressure to a person's abdomen. The fastening and tightening mechanism may be, for instance, a suitable number of straps with hook and loop fastener and buckles. The person tightens the belt around their abdomen, applying sufficient pressure to inhibit their diaphragm from fully expanding, thereby disrupting the person's ability to snore and regulating the person's breaths.

### BRIEF DESCRIPTION OF THE DRAWINGS

These and other features, aspects, and advantages of the present invention will become better understood with reference to the following description, appended claims, and accompanying drawings where:

FIG. 1 is a perspective view of a device for reducing and preventing snoring and sleep apnea, having two sets of straps and buckles;

FIG. 2 is a partial front, perspective view of a person wearing the device;

FIG. 3 is a partial left side, perspective view of a person wearing the device; and

FIG. 4 is a perspective view of an alternative embodiment of a device for reducing and preventing snoring and sleep apnea, having three sets of straps and buckles;

FIG. 5 is a flowchart of the steps of the method of using the device to treat snoring and sleep apnea.

### DESCRIPTION OF THE PREFERRED EMBODIMENT

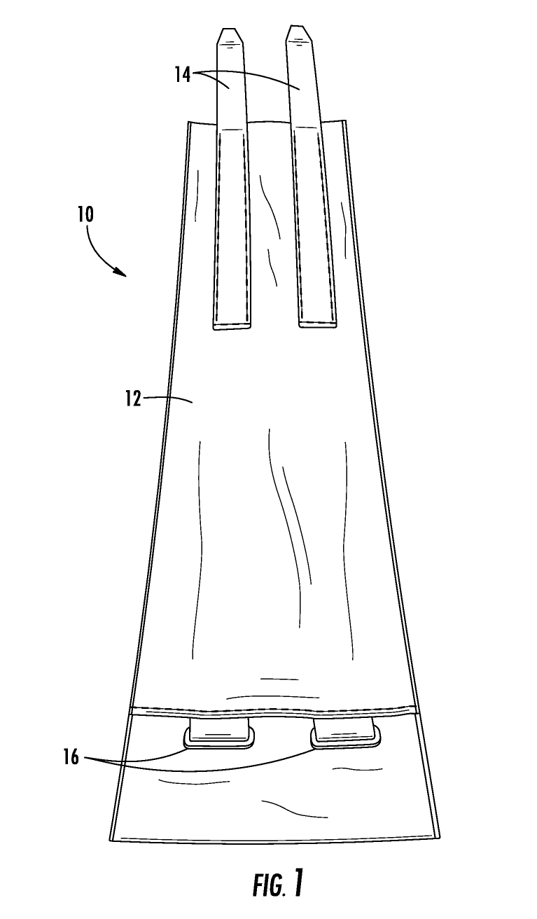

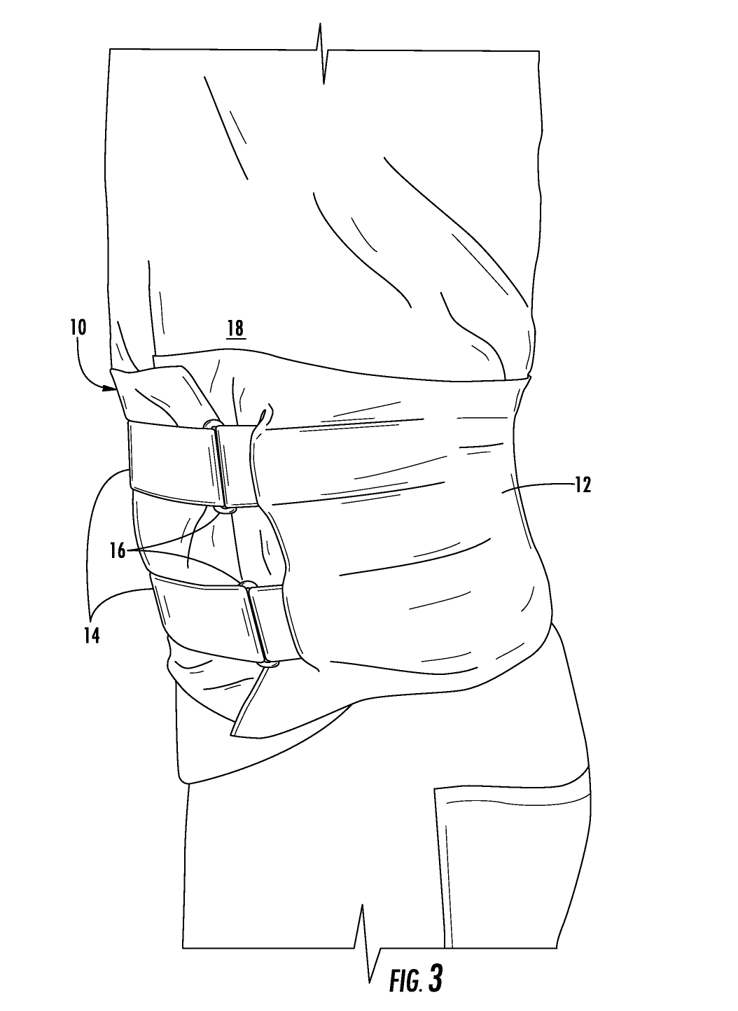

Referring now to FIGS. 1-3, an embodiment of the device for reducing and preventing snoring is shown generally at 10. The device includes a wide cummerbund-style belt 12 of sufficient length to wrap around a person's 18 torso and of sufficient width to overlap the person's 18 abdomen. The belt 12 includes a fastening and tightening mechanism to compress the belt against a person's 18 abdomen, applying specific pressure beneath (lower on the body) a person's 18 diaphragm.

One embodiment of the fastening and tightening mechanism is a strap 14 with hook and loop fastener and a buckle 16. The buckle 16 is located on one end of the belt 12 and the strap 14 is attached to the other end. The strap 14 is attached along the length of the belt 12 and includes hook and loop fasteners configured so that the strap 14 may fasten to itself, once threaded through the buckle 16 the belt 12 is positioned on the person's 18 abdomen.

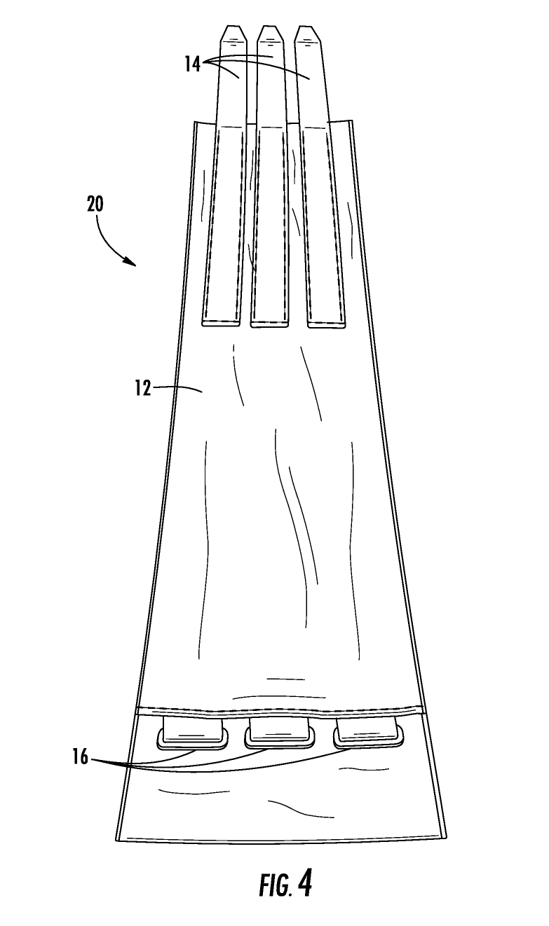

In this embodiment, a pair of straps and buckles is shown adjacent to one another. However, more may be included as shown in FIG. 4. Additionally, although strap 14 with hook and loop fasteners and buckles 16 are shown, other style fastening and tightening mechanisms may be used, including, but not limited to, lacings, buckles with tongues, plastic snap-in buckles, rings, and other closures as are known in the art.

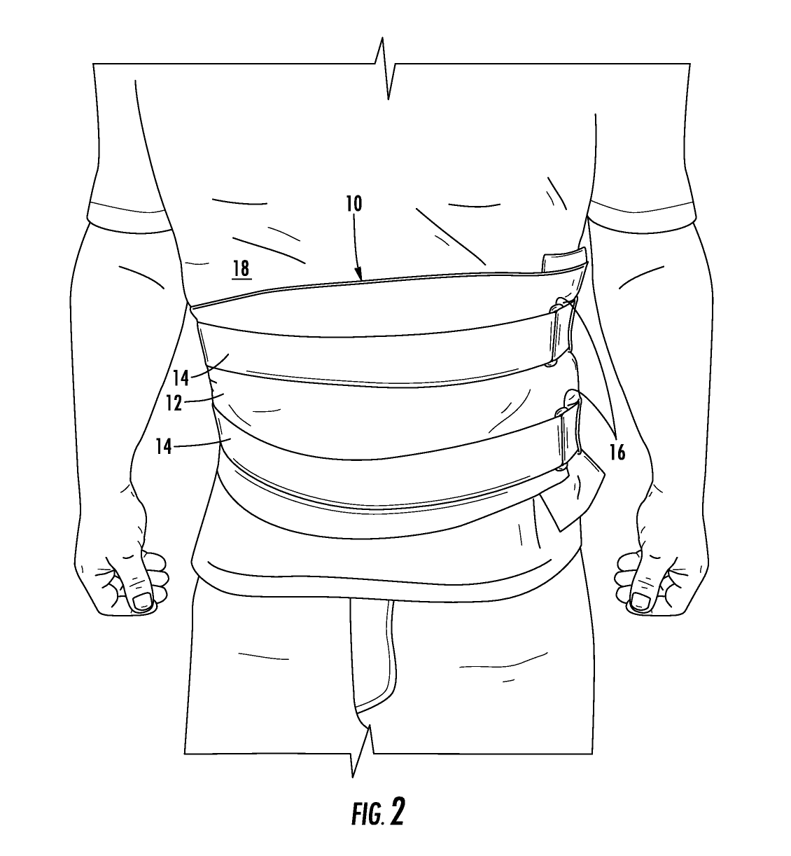

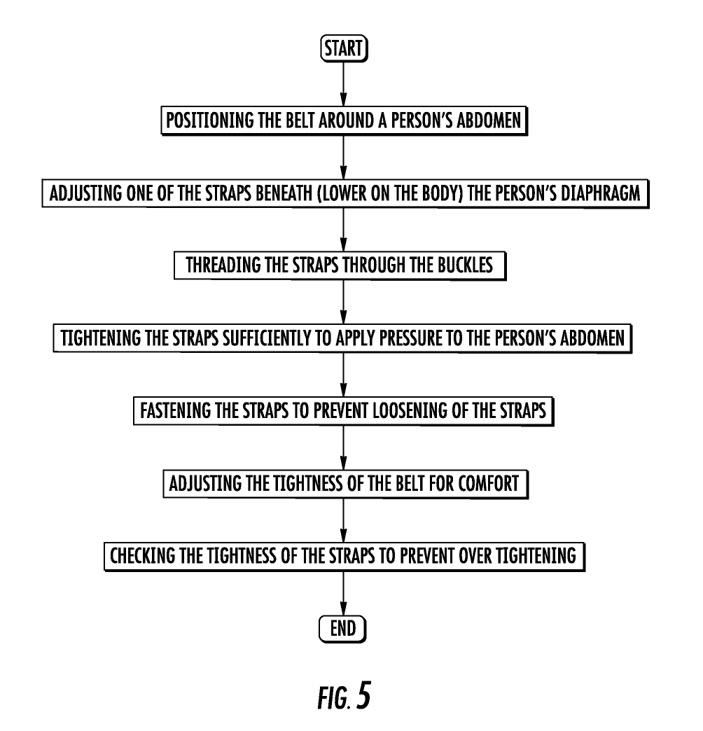

Referring to FIGS. 2, 3 and 5, in use the belt is positioned around a person's 18 abdomen with one of the straps preferably positioned beneath the person's 18 diaphragm. The straps are threaded through the buckles and tightened sufficiently to apply pressure to the person's 18 abdomen, thereby inhibiting the person's 18 ability to take a deep breath, i.e. preventing the diaphragm from being able to fully expand. The straps are fastened via the hook and loop fasteners to prevent loosening of the straps. Because the person's 18 breathing is inhibited, the belt reduces and prevents a person 18 from snoring. The tightness of the belt may be adjusted for comfort. Furthermore, the straps should not be tightened so tight that suffocation or hypoxia becomes a risk.

Referring to FIG. 4, an alternative embodiment of the device is shown generally at 100. The alternative embodiment of the device includes a belt with three strap and buckle sets.

Therefore, it can be seen that device and method of preventing and/or reducing snoring and sleep apnea provides a unique way of managing snoring and sleep apnea without resorting to uncomfortable mouth guards, face masks, or harnesses that prevent sleeping on one's back.

It would be appreciated by those skilled in the art that various changes and modifications can be made to the illustrated embodiments without departing from the spirit of the present invention. All such modifications and changes are intended to be within the scope of the present invention.

What is claimed is:

1. A method of reducing and preventing snoring and sleep apnea, comprising:
positioning a belt around a person's abdomen, the belt of sufficient length to wrap around a person's torso and of sufficient width to overlap the person's abdomen, the belt further including a fastening and tightening mechanism to compress the belt against a person's abdomen;
tightening the belt with the fastening and tightening mechanism, sufficiently to apply pressure to the person's abdomen, applying specific pressure beneath a person's diaphragm; and
fastening the fastening and tightening mechanism to prevent loosening of the belt.

2. The method of claim 1, further comprising adjusting the tightness of the belt to prevent over tightening.

3. The method of claim 1, further comprising adjusting the tightness of the belt for the person's comfort.

4. The method of claim 1, wherein the fastening and tightening mechanism of the belt includes a strap and a buckle, the method further including the step of threading the strap through the buckle.

5. The method of claim 4, wherein the step of positioning the belt around a person's abdomen further comprises positioning the strap on the person's abdomen beneath the person's diaphragm.

6. A device for reducing and preventing snoring and sleep apnea, comprising:
a belt of sufficient length to wrap around a person's torso and of sufficient width to overlap the person's abdomen;
a strap and buckle attached to the belt configured and arranged to tighten the belt;
whereby the belt is positioned around a person's abdomen and the strap and buckle are tightened sufficiently to apply pressure to the person's abdomen, applying specific pressure beneath a person's diaphragm, thereby preventing or reducing snoring of the person.

7. The device of claim 6, wherein there are two strap and buckle sets, spaced apart from each other, each set being individually adjustable and fastenable.

8. The device of claim 7, wherein there is a third strap and buckle set spaced apart from the other two sets, the third set being individually adjustable and fastenable.

9. The device of claim 6, wherein the strap further includes hook and loop fasteners thereon.
